(19) United States
(12) Patent Application Publication
(10) Pub. No.: US 2023/0264010 A1
(43) Pub. Date: Aug. 24, 2023

(54) MEDICAL STOPCOCK ASSEMBLY WITH POSITION DETECTION

(71) Applicant: Becton, Dickinson and Company, Franklin Lakes, NJ (US)

(72) Inventors: John Picot, Landing, NJ (US); Hong Zhu, Glen Rock, NJ (US); Matthew Lee Kolb, Bloomingdale, NJ (US)

(21) Appl. No.: 18/013,392

(22) PCT Filed: Jun. 29, 2021
(86) PCT No.: PCT/US21/39548

§ 371 Date: Dec. 28, 2022

Related U.S. Application Data
(60) Provisional application No. 63/046,334, filed on Jun. 30, 2020.

(51) Int. Cl. A61M 39/22

(52) U.S. Cl. CPC A61M 39/223; A61M 2039/229; A61M 2205/3306; A61M 2205/3327; A61M 2205/3584

(57) ABSTRACT

A medical stopcock assembly with position detection includes a medical stopcock and a sensor. The medical stopcock may include a main body and a valve body. The valve body may define a valve passageway and be moveable relative to the main body between a first position where a first input port and an output port are in fluid communication via the valve passageway, a second position where a second input port and the output port are in fluid communication via the valve passageway, and a third position where the second input port and the first input port are in fluid communication via the valve passageway. An external surface of the valve body may include a position structure. The sensor may be configured to sense the position structure to determine whether the valve body is in the first position, the second position, or the third position.

### CROSS-REFERENCE TO RELATED APPLICATION

The present application claims priority to U.S. Provisional Application Ser. No. 63/046,334, entitled “Medical Stopcock Assembly with Position Detection”, filed Jun. 30, 2020, the entire disclosure of which is hereby incorporated by reference in its entirety.

### BACKGROUND

### 1. Field

The present disclosure relates generally to a medical stopcock and, in some non-limiting embodiments or aspects, to a medical stopcock smart valve assembly including valve position detection.

### 2. Technical Considerations

Stopcocks, such as 3-way stopcocks, include two input ports and one output port. In some configurations, a handle of the stopcock may be operated by a healthcare professional to place the stopcock in three positions, including where a first input port is in fluid communication with the output port, where a second input port is in fluid communication with the output port, and where the first input port is in fluid communication with the second input port. The healthcare professional may manually update a log that tracks a status of the stopcock handle.

### SUMMARY

Accordingly, provided are improved systems, devices, products, apparatus, and/or methods for stopcock position detection.

According to some non-limiting embodiments or aspects, provided is a medical stopcock assembly with position detection including: a medical stopcock including: a main body; a valve body, wherein the valve body defines a valve passageway and is moveable relative to the main body between a first position where a first input port and an output port are in fluid communication via the valve passageway, a second position where a second input port and the output port are in fluid communication via the valve passageway, and a third position where the second input port and the first input port are in fluid communication via the valve passageway, and wherein an external surface of the valve body includes at least one position structure; and at least one sensor configured to sense the at least one position structure to determine whether the valve body is in the first position, the second position, or the third position.

According to some non-limiting embodiments or aspects, provided is a medical stopcock including: a main body; and a valve body, wherein the valve body defines a valve passageway and is moveable relative to the main body between a first position where a first input port and an output port are in fluid communication via the valve passageway, a second position where a second input port and the output port are in fluid communication via the valve passageway, and a third position where the second input port and the first input port are in fluid communication via the valve passageway, and wherein an external surface of the valve body includes at least one position structure.

Further embodiments or aspects are set forth in the following numbered clauses:

Clause 1. A medical stopcock assembly with position detection comprising: a medical stopcock including: a main body; a valve body, wherein the valve body defines a valve passageway and is moveable relative to the main body between a first position where a first input port and an output port are in fluid communication via the valve passageway, a second position where a second input port and the output port are in fluid communication via the valve passageway, and a third position where the second input port and the first input port are in fluid communication via the valve passageway, and wherein an external surface of the valve body includes at least one position structure; and at least one sensor configured to sense the at least one position structure to determine whether the valve body is in the first position, the second position, or the third position.

Clause 2. The medical stopcock assembly of clause 2, wherein a portion of the valve body extends from a top of the main body, wherein the portion of the valve body extending from the top of the main body includes a handle, and wherein the at least one position structure includes at least one recessed portion of the handle.

Clause 3. The medical stopcock assembly of any of clauses 1 and 2, wherein the at least one recessed portion of the handle includes a first recessed portion and a second recessed portion, and wherein the first recessed portion and the second recessed portion are spaced apart about a circumference of the valve body in a same radial plane of the valve body.

Clause 4. The medical stopcock assembly of any of clauses 1-3, wherein the first recessed portion and the second recessed portion are spaced apart 90 degrees about the circumference of the valve body.

Clause 5. The medical stopcock assembly of any of clauses 1-4, wherein the at least one sensor includes a first sensor and a second sensor, and wherein the first sensor and the second sensor are in the same radial plane of the valve body and a same tangential plane of the valve body.

Clause 6. The medical stopcock assembly of any of clauses 1-5, wherein a first portion of the valve body extends from a top of the main body, wherein the first portion of the valve body extending from the top of the main body includes a handle, wherein a second portion of the valve body extends from a bottom of the main body opposite the top of the main body, wherein the second portion of the valve body includes the at least one position structure, and wherein the at least one position structure includes at least one recessed portion of the valve body.

Clause 7. The medical stopcock assembly of any of clauses 1-6, wherein the at least one recessed portion includes a first recessed portion and a second recessed portion, and wherein the first recessed portion and the second recessed portion are spaced apart about a circumference of the valve body and in an axial direction of the valve body.

Clause 8. The medical stopcock assembly of any of clauses 1-7, wherein the first recessed portion and the second recessed portion are spaced apart 90 degrees about the circumference of the valve body.

Clause 9. The medical stopcock assembly of any of clauses 1-8, wherein the at least one sensor includes a first sensor and a second sensor, and wherein the first sensor is located above the second sensor in the axial direction of the valve body in a same tangential plane of the valve body.

Clause 10. The medical stopcock assembly of any of clauses 1-9, wherein the at least one recessed portion includes a single recessed portion.

Clause 11. The medical stopcock assembly of any of clauses 1-10, wherein the at least one sensor includes a first sensor and a second sensor, and wherein the first sensor and the second sensor are spaced apart about the circumference of the valve body in the same radial plane of the valve body.

Clause 12. The medical stopcock assembly of any of clauses 1-11, wherein the first sensor and the second sensor are spaced apart 90 degrees about the circumference of the valve body in the same radial plane of the valve body.

Clause 13. The medical stopcock assembly of any of clauses 1-12, wherein a first portion of the valve body extends from a top of the main body, wherein the first portion of the valve body extending from the top of the main body includes a handle, wherein a second portion of the valve body extends from a bottom of the main body opposite the top of the main body, wherein an end of the second portion of the valve body opposite the bottom of the main body includes a disc, wherein a face of the disc facing opposite a direction toward the top of the main body includes the at least one position structure, and wherein the at least one position structure includes at least one recessed portion of the disc.

Clause 14. The medical stopcock assembly of any of clauses 1-13, further comprising: a circuit board including a face facing the face of the disc facing opposite the direction toward the top of the main body, wherein the face of the circuit board includes the at least one sensor.

Clause 15. The medical stopcock assembly of any of clauses 1-14, wherein the at least one sensor includes a first sensor and a second sensor, and wherein the first sensor and the second sensor are spaced apart 90 degrees on a plane of the circuit board.

Clause 16. The medical stopcock assembly of any of clauses 1-15, wherein the at least one sensor is configured to determine a distance to the external surface of the valve body to determine whether the valve body is in the first position, the second position, or the third position.

Clause 17. The medical stopcock assembly of any of clauses 1-16, wherein the at least one sensor includes at least one of a proximity detector, an image sensor, a time-of-flight sensor, or any combination thereof.

Clause 18. The medical stopcock assembly of any of clauses 1-17, further comprising: a housing including the at least one sensor, wherein the housing holds the at least one sensor in a fixed position when the valve body moves relative to the main body.

Clause 19. The medical stopcock assembly of any of clauses 1-18, wherein the housing is attached to the main body.

Clause 20. The medical stopcock assembly of any of clauses 1-19, wherein the housing includes a wireless communication device configured to communicate, to a computing device, sensor data sensed by the at least one sensor.

Clause 21. A medical stopcock comprising: a main body; and a valve body, wherein the valve body defines a valve passageway and is moveable relative to the main body between a first position where a first input port and an output port are in fluid communication via the valve passageway, a second position where a second input port and the output port are in fluid communication via the valve passageway, and a third position where the second input port and the first input port are in fluid communication via the valve passageway, and wherein an external surface of the valve body includes at least one position structure.

Clause 22. The medical stopcock of clause 21 comprising the medical stopcock of any of clauses 1-20.

These and other features and characteristics of the present disclosure, as well as the methods of operation and functions of the related elements of structures and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of limits. As used in the specification and the claims, the singular form of “a,” “an,” and “the” include plural referents unless the context clearly dictates otherwise.

### BRIEF DESCRIPTION OF THE DRAWINGS

Additional advantages and details of embodiments or aspects of the present disclosure are explained in greater detail below with reference to the exemplary embodiments that are illustrated in the accompanying schematic figures, in which:

FIGS. 1A-1D are sectional views of non-limiting embodiments or aspects of a stopcock illustrating four different orientations for a valve body movable relative to a main body;

FIG. 2 is a diagram of non-limiting embodiments or aspects of components of a housing;

FIG. 3A is a side view of non-limiting embodiments or aspects of a medical stopcock assembly;

FIG. 3B is a top view of non-limiting embodiments or aspects of a medical stopcock assembly;

FIG. 3C is a top cut-away view of non-limiting embodiments or aspects of a medical stopcock assembly;

FIG. 3D is another top cut-away view of non-limiting embodiments or aspects of a medical stopcock assembly;

FIG. 4A is a side view of non-limiting embodiments or aspects of a medical stopcock assembly

FIG. 4B is a bottom cut-away view of non-limiting embodiments or aspects of a medical stopcock assembly;

FIG. 4C is a partially exploded perspective view of non-limiting embodiments or aspects of a medical stopcock assembly;

FIG. 4D is a perspective view of non-limiting embodiments or aspects of a medical stopcock assembly;

FIG. 5A is a perspective view of non-limiting embodiments or aspects of a medical stopcock assembly;

FIG. 5B is a partially exploded perspective view of non-limiting embodiments or aspects of a medical stopcock assembly;

FIG. 5C is a bottom cut-away view of non-limiting embodiments or aspects of a medical stopcock assembly;

FIG. 6A is a partially exploded side view of non-limiting embodiments or aspects of a medical stopcock assembly;

FIG. 6B is a perspective view of non-limiting embodiments or aspects of a medical stopcock assembly; and

FIG. 6C is a partially exploded perspective view of non-limiting embodiments or aspects of a medical stopcock assembly.

### DETAILED DESCRIPTION

It is to be understood that the present disclosure may assume various alternative variations and step sequences, except where expressly specified to the contrary. It is also to be understood that the specific devices and processes illustrated in the attached drawings, and described in the following specification, are simply exemplary and non-limiting embodiments or aspects. Hence, specific dimensions and other physical characteristics related to the embodiments or aspects disclosed herein are not to be considered as limiting.

For purposes of the description hereinafter, the terms “upper”, “lower”, “right”, “left”, “vertical”, “horizontal”, “top”, “bottom”, “lateral”, “longitudinal”, and derivatives thereof shall relate to the invention as it is oriented in the drawing figures. However, it is to be understood that the invention may assume various alternative variations, except where expressly specified to the contrary. It is also to be understood that the specific devices illustrated in the attached drawings, and described in the following specification, are simply exemplary aspects of the invention. Hence, specific dimensions and other physical characteristics related to the aspects disclosed herein are not to be considered as limiting. All numbers and ranges used in the specification and claims are to be understood as being modified in all instances by the term “about”. By “about” is meant plus or minus twenty-five percent of the stated value, such as plus or minus ten percent of the stated value. However, this should not be considered as limiting to any analysis of the values under the doctrine of equivalents.

Unless otherwise indicated, all ranges or ratios disclosed herein are to be understood to encompass the beginning and ending values and any and all subranges or sub-ratios subsumed therein. For example, a stated range or ratio of “1 to 10” should be considered to include any and all subranges or sub-ratios between (and inclusive of) the minimum value of 1 and the maximum value of 10; that is, all subranges or sub-ratios beginning with a minimum value of 1 or more and ending with a maximum value of 10 or less. The ranges and/or ratios disclosed herein represent the average values over the specified range and/or ratio.

The terms “first”, “second”, and the like are not intended to refer to any particular order or chronology, but refer to different conditions, properties, or elements.

No aspect, component, element, structure, act, step, function, instruction, and/or the like used herein should be construed as critical or essential unless explicitly described as such. Also, as used herein, the articles “a” and “an” are intended to include one or more items, and may be used interchangeably with “one or more” and “at least one.” Furthermore, as used herein, the term “set” is intended to include one or more items (e.g., related items, unrelated items, a combination of related and unrelated items, etc.) and may be used interchangeably with “one or more” or “at least one.” Where only one item is intended, the term “one” or similar language is used. Also, as used herein, the terms “has,” “have,” “having,” or the like are intended to be open-ended terms. Further, the phrase “based on” is intended to mean “based at least in partially on” unless explicitly stated otherwise.

As used herein, the terms “communication” and “communicate” refer to the receipt or transfer of one or more signals, messages, commands, or other type of data. For one unit (e.g., any device, system, or component thereof) to be in communication with another unit means that the one unit is able to directly or indirectly receive data from and/or transmit data to the other unit. This may refer to a direct or indirect connection that is wired and/or wireless in nature. Additionally, two units may be in communication with each other even though the data transmitted may be modified, processed, relayed, and/or routed between the first and second unit. For example, a first unit may be in communication with a second unit even though the first unit passively receives data and does not actively transmit data to the second unit. As another example, a first unit may be in communication with a second unit if an intermediary unit processes data from one unit and transmits processed data to the second unit. It will be appreciated that numerous other arrangements are possible.

It will be apparent that systems and/or methods, described herein, can be implemented in different forms of hardware, software, or a combination of hardware and software. The actual specialized control hardware or software code used to implement these systems and/or methods is not limiting of the implementations. Thus, the operation and behavior of the systems and/or methods are described herein without reference to specific software code, it being understood that software and hardware can be designed to implement the systems and/or methods based on the description herein.

Some non-limiting embodiments or aspects are described herein in connection with thresholds. As used herein, satisfying a threshold may refer to a value being greater than the threshold, more than the threshold, higher than the threshold, greater than or equal to the threshold, less than the threshold, fewer than the threshold, lower than the threshold, less than or equal to the threshold, equal to the threshold, etc.

As used herein, the term “computing device” or “computer device” may refer to one or more electronic devices that are configured to directly or indirectly communicate with or over one or more networks. The computing device may be a mobile device, a desktop computer, or the like. Furthermore, the term “computer” may refer to any computing device that includes the necessary components to receive, process, and output data, and normally includes a display, a processor, a memory, an input device, and a network interface. An “application” or “application program interface” (API) refers to computer code or other data sorted on a computer-readable medium that may be executed by a processor to facilitate the interaction between software components, such as a client-side front-end and/or server-side back-end for receiving data from the client. An “interface” refers to a generated display, such as one or more graphical user interfaces (GUIs) with which a user may interact, either directly or indirectly (e.g., through a keyboard, mouse, touchscreen, etc.).

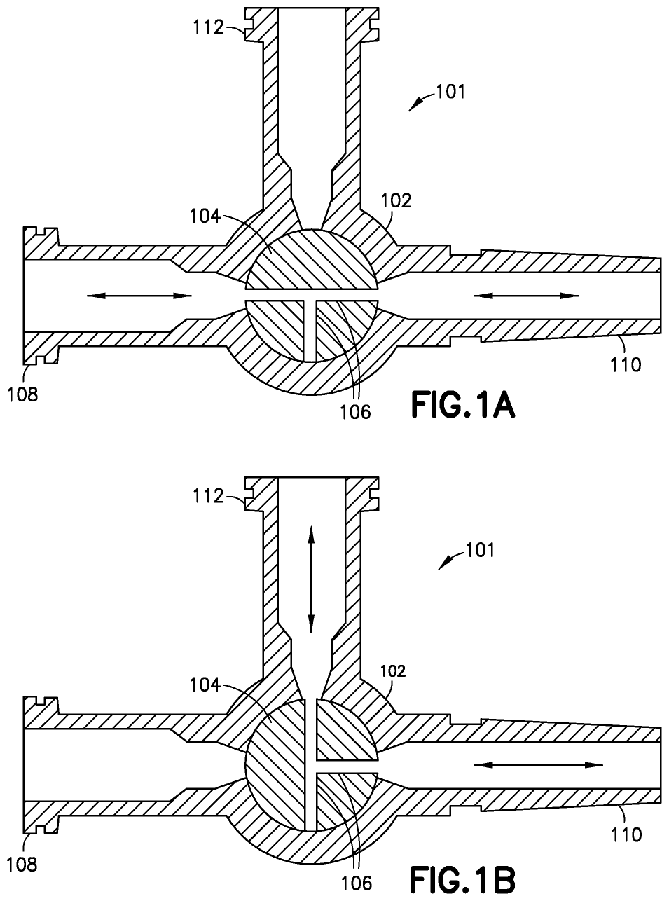

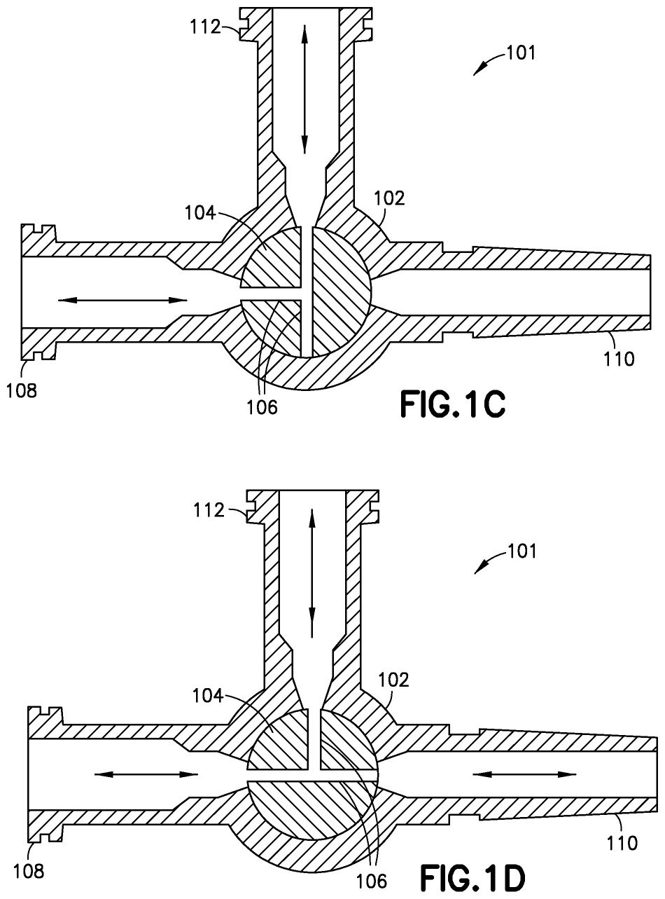

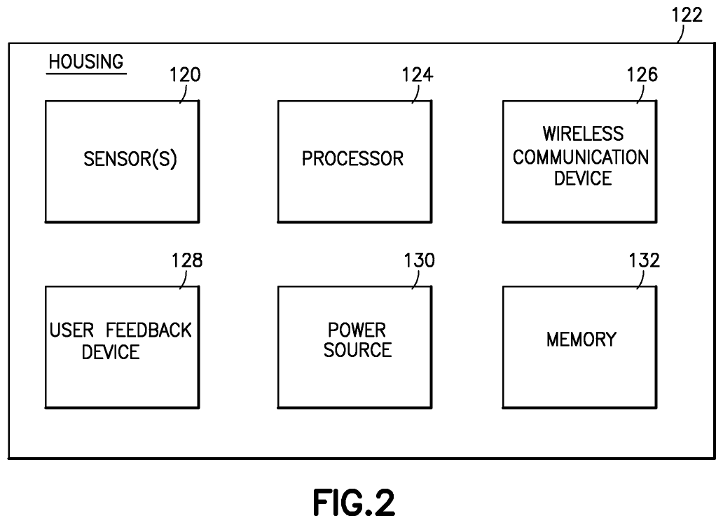

Referring to FIGS. 1A-1D, 2, 3A-3D, 4A-4D, 5A-5C, and 6A-6C, medical stopcock assembly 100 may include a medical stopcock 101 and/or a sensor(s) 120. Medical stopcock 101 may include main body 102 and/or valve body 104. Valve body 104 may define valve passageway 106 and be moveable relative to main body 102 between a first position (FIG. 1A) where first input port 108 and output port 110 are in fluid communication via valve passageway 106, a second position (FIG. 1B) where second input port 112 and output port 110 are in fluid communication via valve passageway 106, and/or a third position (FIG. 10) where second input port 112 and first input port 108 are in fluid communication via valve passageway 106. In some non-limiting embodiments or aspects, valve body 104 may be moveable relative to main body 102 between the first position (FIG. 1A), the second position (FIG. 1B), the third position (FIG. 10), and/or a fourth position (FIG. 1D) where each of first input port 108, output port 110, and second input port 112 are in fluid communication via valve passageway 106.

Main body 102 may define a valve opening between top 103a of main body 102 and bottom 103b of main body 102, and valve body 104 may re received within the valve opening. For example, the valve opening of main body 102 may include a cylinder shaped opening, and valve body 103 may have a cylinder shape configured to be received in the cylinder shaped opening of main body 102. As an example, valve body 104 and the valve opening of main body 102 may have a tight fit, which may require a user to use handle 114 to move valve body 104. As another example, valve body 104 and the valve opening of the main body 102 may have a looser fit. A portion of valve body 104 may extend from top 103a of main body 102, and the portion of valve body 104 extending from top 103a of main body 102 may include a handle 114. Rotation of handle 114 may be configured to move valve body 104 relative to main body 102. In some non-limiting embodiments or aspects, second input port 112 may include a syringe port configured to connect to a syringe. In some non-limiting embodiments or aspects, medical stopcock assembly 100 may be utilized in connection with a flow sensor system, such as the flow sensor system disclosed in U.S. Pat. No. 9,970,794, which is hereby incorporated by reference in its entirety.

In some non-limiting embodiments or aspects, main body 102 may include first input port 108, output port 110, and second input port 112. For example, FIGS. 1A-1D, 3A-3D, 4A-4D, 5A-5C, and 6A-6C show each of first input port 108, output port 110, and second input port 112 extending from main body 102. However, non-limiting embodiments or aspects are not limited thereto, and in some non-limiting embodiments or aspects main body 102 may include first input port 108 and output port 110, and valve body 104 may include second input port 112. For example, medical stopcock 101 may include a medical stopcock as disclosed in U.S. Provisional Patent Application No. 62/966,086, filed on Jan. 27, 2020, which is hereby incorporated by reference in its entirety, in which rotation of a syringe port is configured to move a valve body relative to a main body.

External surface 116 of valve body 104 may include position structure(s) 118. For example, position structure(s) 118 may include at least one recessed portion (e.g., a recessed portion, an opening, a pocket, a depression, a indent, etc.) of valve body 102. As an example, position structure(s) 118 may act as a flag or shutter that can be sensed by an external sensor or mechanism to determine the position of valve body 104. In such an example, external surface 116 may be located on an outside of valve body 104 external to main body 102 and/or valve passageway 106. In some non-limiting embodiments or aspects, position structure(s) 118 may include at least one structure other than a recessed portion, such as, an extended portion (e.g., a bump, a protrusion, etc.), a coloring and/or pattern different than a coloring of external surface 116 of valve body 104, and/or the like.

Sensor(s) 120 may be configured to sense position structure(s) 118 to determine whether valve body 104 is in the first position, the second position, or the third position (or the fourth position). In some non-limiting embodiments or aspects, sensor(s) 120 is configured to determine a distance to external surface 116 of valve body 104 to determine whether valve body 104 is in the first position, the second position, or the third position (or the further position). For example, sensor(s) 120 (e.g., sensor 120, first sensor 120a, second sensor 120b, etc.) may include at least one of a proximity detector, an image sensor, a time-of-flight sensor, or any combination thereof. In such an example, sensor(s) 120 may determine, based on a detected distance to external surface 116 of valve body 104, a location of position structure(s) 118 (e.g., a location of position structure(s) 118 relative to sensor(s) 120 and/or main body 102, whether position structure(s) 118 is detected or not by sensor(s) 120, etc.), and determine, based on the determined location (or detection) of position structure(s) 118, whether valve body 104 is in the first position, the second position, or the third position (or the fourth position). As an example, as described in more detail herein below, each of the first position, the second position, and the third position of valve body 104 may be associated with or correspond to a different determined location (or detection combination) of position structure(s) 118.

Medical stopcock assembly 100 may include housing 122 including sensor(s) 120. For example, housing 122 may house and/or hold sensor(s) 120 in a fixed position when valve body 104 moves relative to main body 102. As an example, housing 122 may be attached to (e.g., removably attached to, integrated with, etc.) main body 102 such that valve body 104 moves relative to main body 102 and housing 122. In some non-limiting embodiments or aspects, housing 122 (and/or sensor(s) 120) may be a reusable housing/sensor(s) that is configured to be attached to and used with a first stopcock, removed from the first stopcock after user, and subsequently attached to and used with a different stopcock. In some non-limiting embodiments or aspects, housing 122 (and/or sensor(s) 120) may be a disposable housing/sensors(s) that is configured to be disposed of with a stopcock after use with that single stopcock.

In some non-limiting embodiments or aspects, housing 122 may include, be integrated in, and/or formed by a flow sensor and/or a flow sensor base of a flow sensor system, such as the flow sensor system disclosed in U.S. Pat. No. 9,970,794, which is hereby incorporated by reference in its entirety. For example, a flow sensor may be a disposable and/or consumable device which can be spliced in-line with an IV fluid path. As an example, a housing of the flow sensor may include medical stopcock 101 held and/or housed therein and/or attached in-line therewith in the IV fluid path. For example, a compatible flow sensor base may include and/or house a reader device with sensor(s) 120 configured for stopcock position detection, and when the flow sensor is mounted to the flow sensor base, position structure(s) 118 may be held in a desired and/or predetermined position with respect to (e.g., in view of, etc.) sensor(s) 120. As an example, the flow sensor base may hold sensor(s) 120 in a fixed position relative to the flow sensor housing and/or medical stopcock 101 (e.g., in a fixed position when valve body 104 moves relative to main body 102, etc.). In such an example, a housing of the flow sensor base may include one or more printed circuit board assemblies (PCBAs), and sensor(s) 120 may be assembled onto the one or more PCBAs, which may be mounted in the housing of the flow sensor base with other components, such as a battery, a bar code scanner, a microprocessor, and/or the like. In some non-limiting embodiments or aspects, the housing of the flow sensor base may be a portable device including an enclosure (e.g., a plastic enclosure, etc.) that holds the components thereof inside the enclosure. The housing of the flow sensor base may include a transparent window (e.g., a glass window, a transparent plastic opening, etc.) positioned with respect to sensor(s) 120 (e.g., if sensor(s) include an optical sensor, etc.) to provide sensor(s) 120 with a view of medical stopcock 101 when the flow sensor is mounted to the flow sensor base.

In some non-limiting embodiments or aspects, housing 122 may further include a processor 124, a wireless communication device 126, a user feedback device 128, a power source 130, and/or a memory 132.

Processor 124 may be programmed and/or configured to determine, based on a signal or sensor data received from sensor(s) 120, whether valve body 104 is in the first position, the second position, or the third position (or the fourth position). In some non-limiting embodiments or aspects, processor 124 may include a low power microcontroller unit (MCU). Processor 124 may store information associated with a position of valve body 104 in memory 132.

Wireless communication device 126 may be configured to wirelessly communicate with an external computing device (not shown). For example, wireless communication device 126 may be configured to communicate information associated with a position of valve body 104 to the external computing device. In some non-limiting embodiments or aspects, wireless communication device 126 includes one or more computing devices, chips, contactless transmitters, contactless transceivers, NFC transmitters, RFID transmitters, contact based transmitters, Bluetooth Transceivers® and/or the like that enables wireless communication device 126 to receive information directly from and/or communicate information directly to the external computing device via a short range wireless communication connection (e.g., a communication connection that uses NFC protocol, a communication connection that uses Radio-frequency identification (RFID), a communication connection that uses a Bluetooth® wireless technology standard, and/or the like).

User feedback device 128 may be configured to provide an indication associated with a position of valve body 104 to a user. For example, user feedback device 128 may include at least one of the following: a display, a light-emitting diode (LED), an audio output device (e.g., a buzzer, a speaker, etc.), or any combination thereof.

Power source 130 may be configured to power sensor 120, processor 124, wireless communication device 126, user feedback device 128, and/or memory 130. For example, power source 130 may include a battery (e.g., a rechargeable battery, a disposable battery, etc.).

Memory 130 may be configured to store information associated with a position of valve body 104. In some non-limiting embodiments or aspects, memory 130 may store an identifier associated with stopcock 101, which may be associated with the information associated with the position of valve body 104. In some non-limiting embodiments or aspects, memory 130 may store a current position of valve body 104 and/or a log of positions of valve body 104 over a period of time.

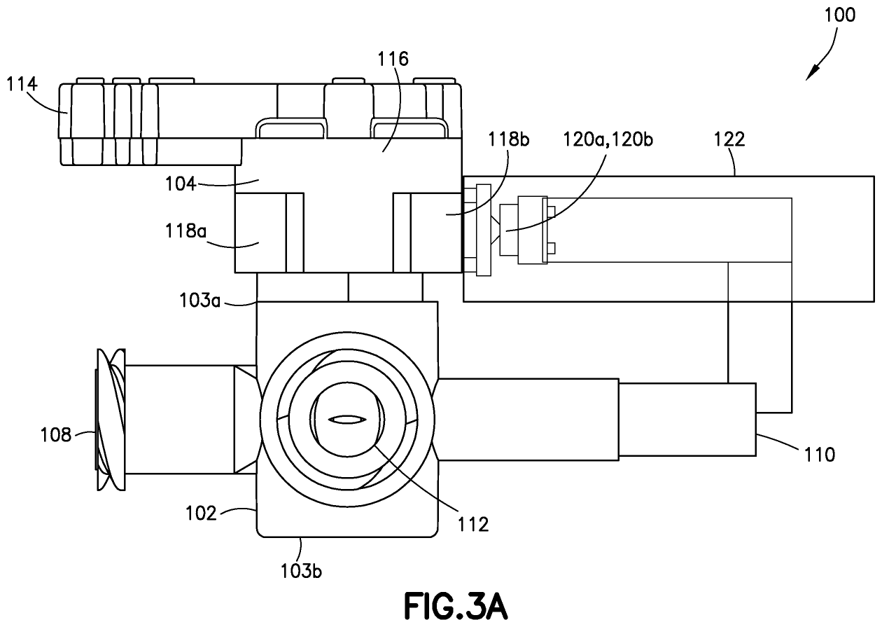

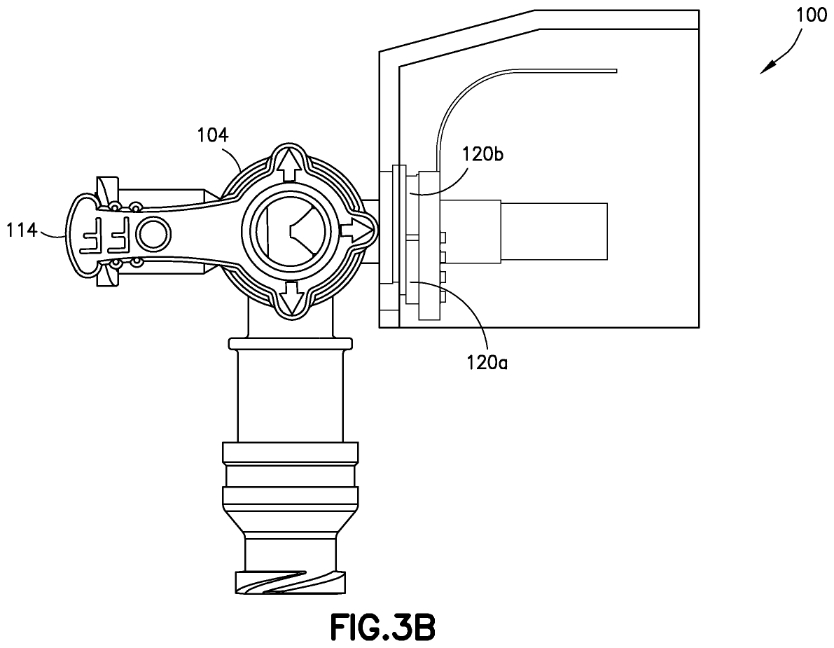

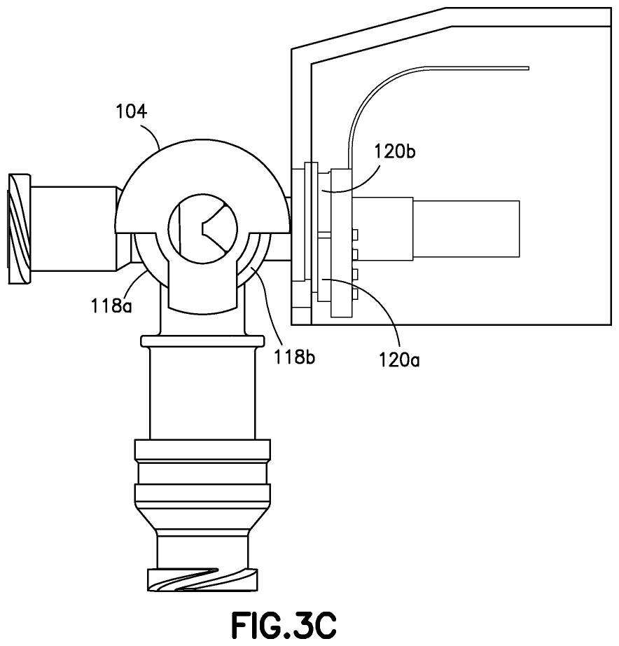

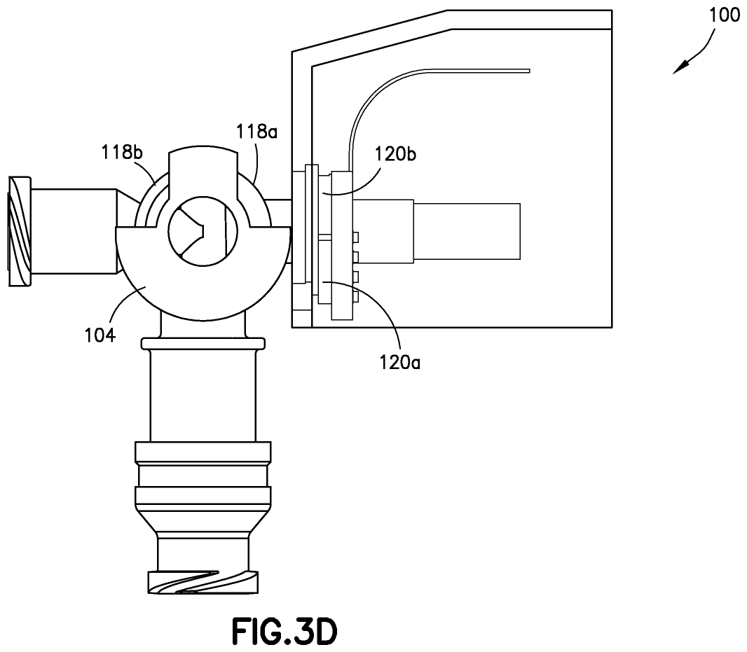

Referring now to FIGS. 3A-3D, in some non-limiting embodiments or aspects, a portion of valve body 104 extends from top 103a of main body 102, the portion of valve body 104 extending from top 103a of main body 102 includes handle 114, and position structure(s) 118 includes at least one recessed portion of handle 114. For example, the at least one recessed portion of handle 114 may include first recessed portion 118a and second recessed portion 118b. As an example, first recessed portion 118a and second recessed portion 118b may be spaced apart about a circumference of valve body 104 in a same radial plane of valve body 104. In such an example, first recessed portion 118a and second recessed portion 118b may spaced apart 90 degrees about the circumference of valve body 104. In such an example, sensor(s) 120 may include first sensor 102a and second sensor 120b. For example, first sensor 120a and second sensor 120b may be in the same radial plane of valve body 104 and a same tangential plane of valve body 104.

Depending on a position of valve body 104 relative to main body 102, first sensor 120a may detect first recessed portion 118a, second recessed portion 118b, or a portion of external surface 116 of valve body 104 without a recessed portion, and second sensor 120b may detect first recessed portion 118a, second recessed portion 118b, or a portion of external surface 116 of valve body 104 without a recessed portion. For example, unique combinations of detections by first sensor 120a and second sensor 120b of first recessed portion 118a, second recessed portion 118b, and/or a portion of external surface 116 of valve body 104 without a recessed portion may be associated with or correspond to the first position, the second position, or the third position (or the fourth position) of valve body 104 (e.g., FIG. 3C may show recessed portions 118a and 118b in a position corresponding to the second position described herein), and first sensor 120a and second sensor 120b (and/or processor 124, and/or the external computing device) may determine based on a detected unique combination a position of valve body 104 in the first position, the second position, or the third position (or the fourth position).

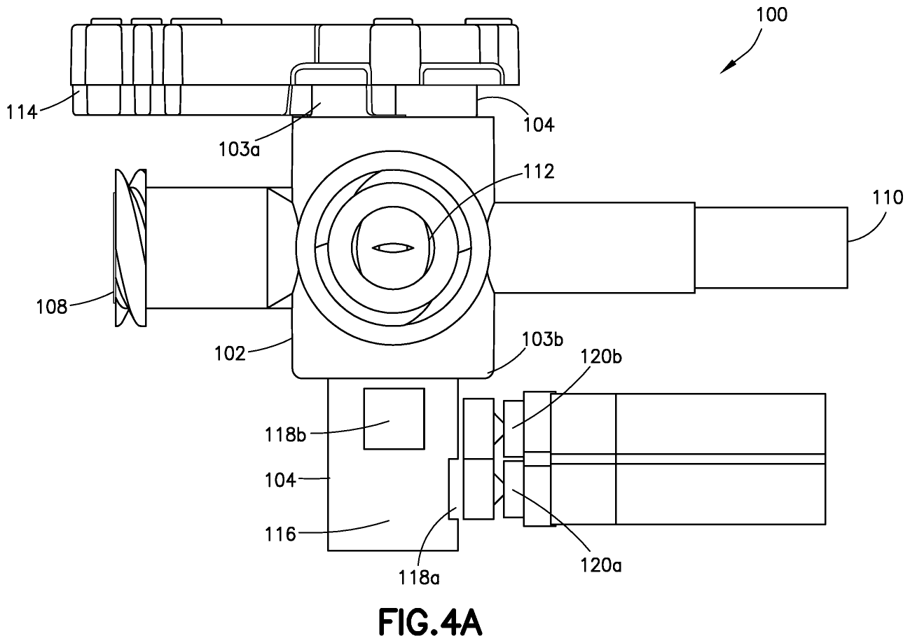

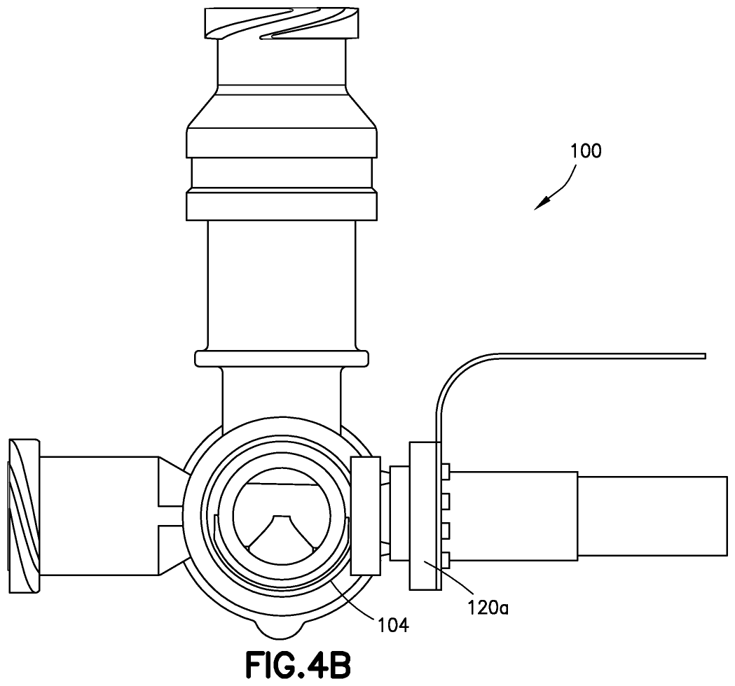

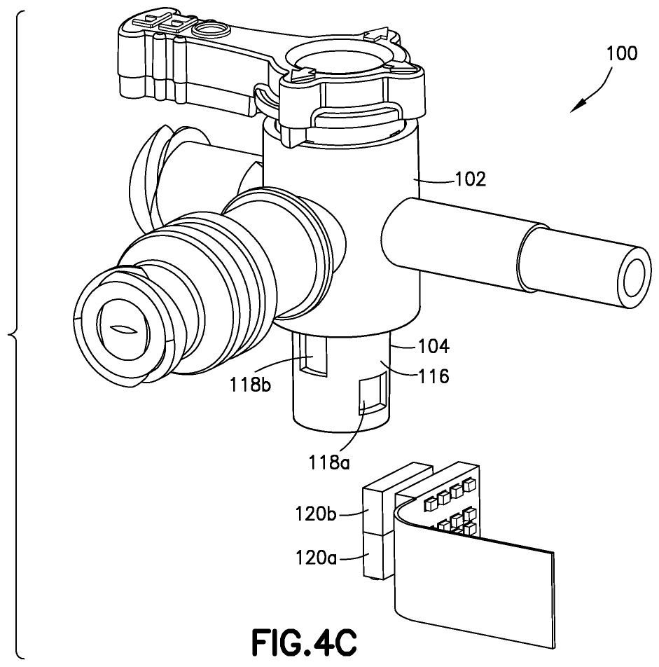

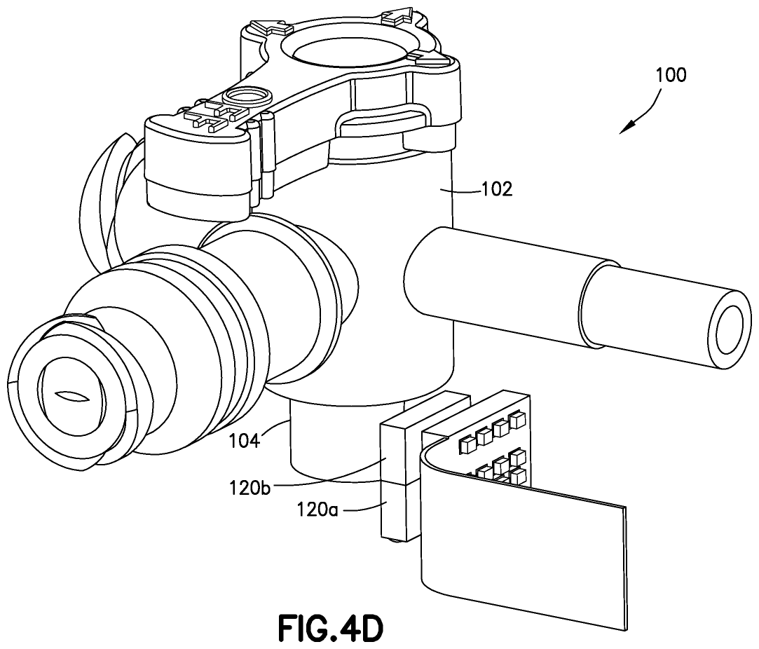

Referring now to FIGS. 4A-4D, in some non-limiting embodiments or aspects, a first portion of valve body 104 extends from top 103a of main body 102, the first portion of valve body 104 extending from top 103a of main body 102 includes handle 114, a second portion of valve body 104 extends from bottom 103b of main body 102 opposite top 103a of main body 102, the second portion of valve body 104 includes position structure(s) 118, position structure(s) 118 includes at least one recessed portion of valve body 104, the at least one recessed portion includes first recessed portion 118a and second recessed portion 118b, and/or first recessed portion 118a and second recessed portion 118b are spaced apart about a circumference of valve body 104 and in an axial direction of valve body 104. For example, first recessed portion 118a and second recessed portion 118b may be spaced apart 90 degrees about the circumference of the valve body 104. In such an example, sensor(s) 120 may include first sensor 120a and second sensor 120b, and second sensor 120b may be located above first sensor 120a in the axial direction of valve body 104 in a same tangential plane of valve body 104.

Depending on a position of valve body 104 relative to main body 102, first sensor 120a may detect first recessed portion 118a or a portion of external surface 116 of valve body 104 without a recessed portion, and second sensor 120b may detect second recessed portion 118b or a portion of external surface 116 of valve body 104 without a recessed portion. For example, unique combinations of detections by first sensor 120a and second sensor 120b of first recessed portion 118a, second recessed portion 118b, and/or a portion of external surface 116 of valve body 104 without a recessed portion may be associated with or correspond to the first position, the second position, or the third position (or the fourth position) of valve body 104 (e.g., FIG. 4A may show recessed portions 118a and 118b in a position corresponding to the second position described herein), and first sensor 120a and second sensor 120b (and/or processor 124, and/or the external computing device) may determine based on a detected unique combination a position of valve body 104 in the first position, the second position, or the third position (or the fourth position).

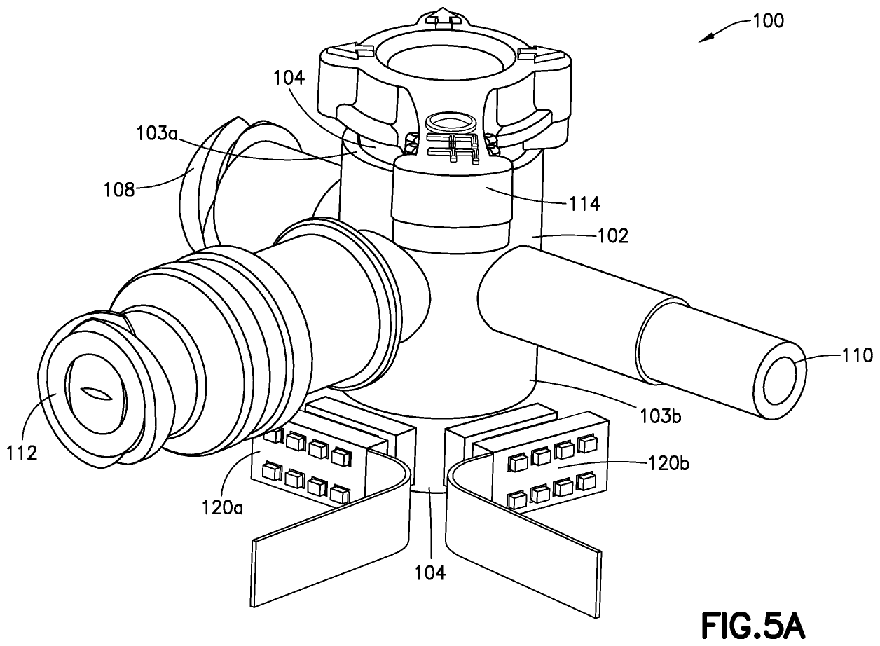

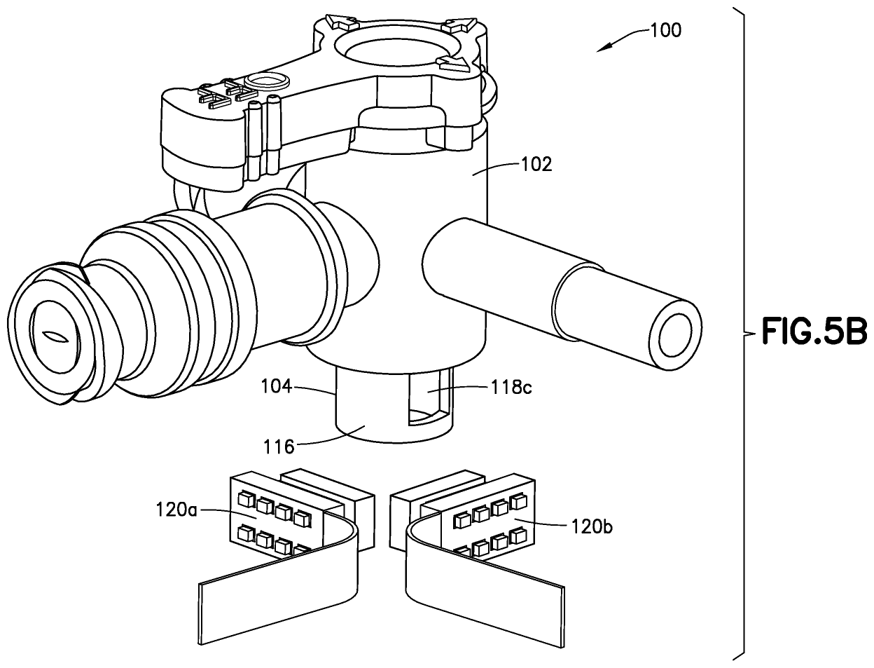

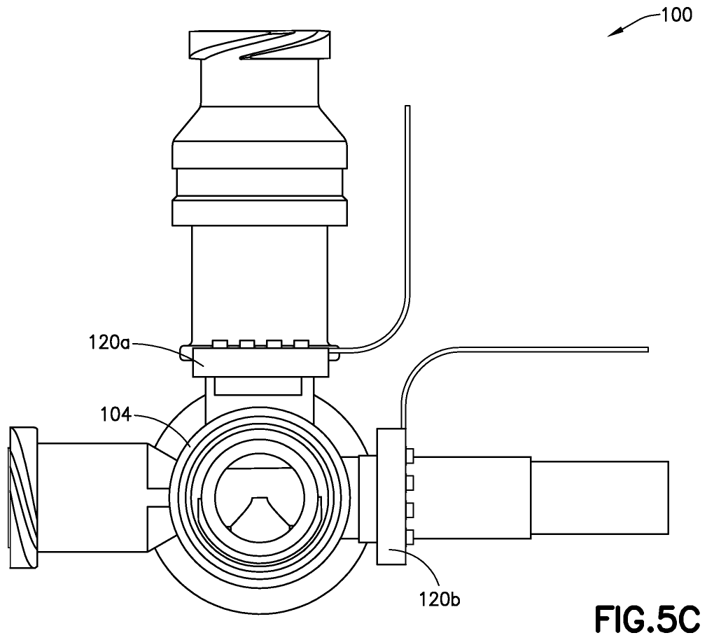

Referring now to FIGS. 5A-5D, in some non-limiting embodiments or aspects, a first portion of valve body 104 extends from top 103a of main body 102, the first portion of valve body 104 extending from top 103a of main body 102 includes handle 114, a second portion of valve body 104 extends from bottom 103b of main body 102 opposite top 103a of main body 102, the second portion of valve body 104 includes position structure(s) 118, position structure(s) 118 includes at least one recessed portion of valve body 104, and/or the at least one recessed portion includes single recessed portion 118c. In such an example, sensor(s) 120 may include first sensor 120a and second sensor 120b, and first sensor 120a and second sensor 120b may be spaced apart about a circumference of valve body 104 in the same radial plane of valve body 104. For example, first sensor 120a and second sensor 120b may be spaced apart 90 degrees about the circumference of valve body 104 in the same radial plane of valve body 104.

Depending on a position of valve body 104 relative to main body 102, first sensor 120a may detect single recessed portion 118c or a portion of external surface 116 of valve body 104 without a recessed portion, and second sensor 120b may detect single recessed portion 118c or a portion of external surface 116 of valve body 104 without a recessed portion. For example, unique combinations of detections by first sensor 120a and second sensor 120b of single recessed portion 118c and/or a portion of external surface 116 of valve body 104 without a recessed portion may be associated with or correspond to the first position, the second position, or the third position (or the fourth position) of valve body 104 (e.g., FIG. 5B may show single recessed portion 118c in a position corresponding to the first position described herein), and first sensor 120a and second sensor 120b (and/or processor 124, and/or the external computing device) may determine based on a detected unique combination a position of valve body 104 in the first position, the second position, or the third position (or the fourth position).

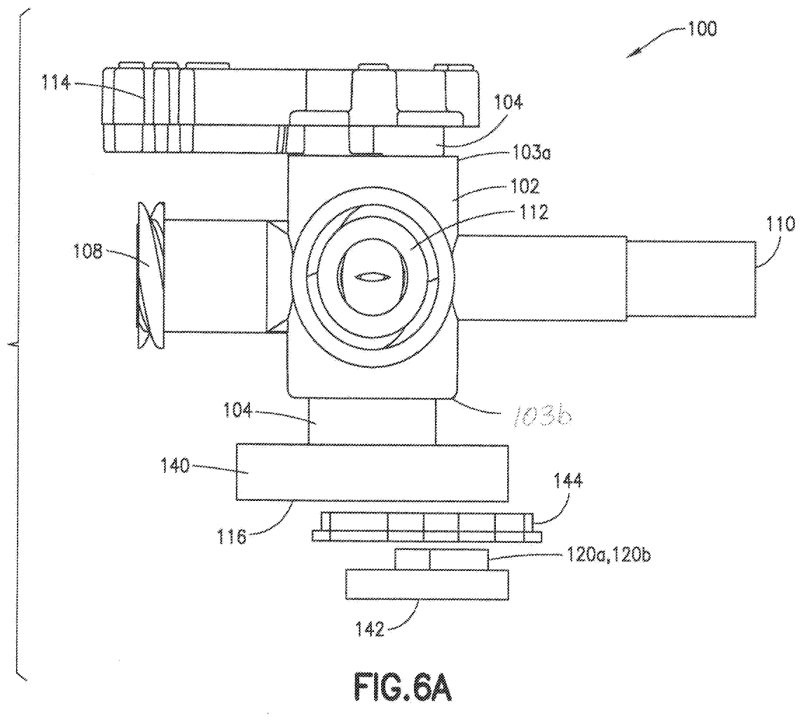

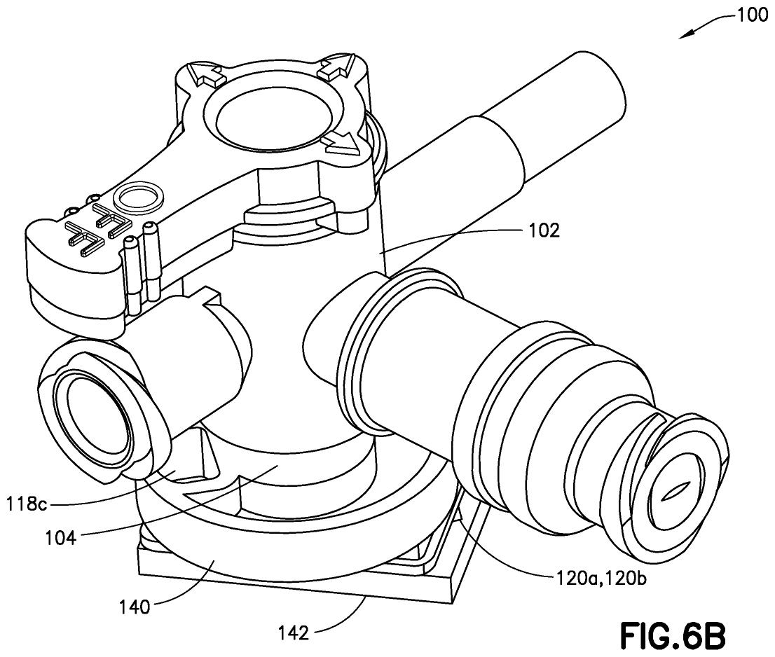

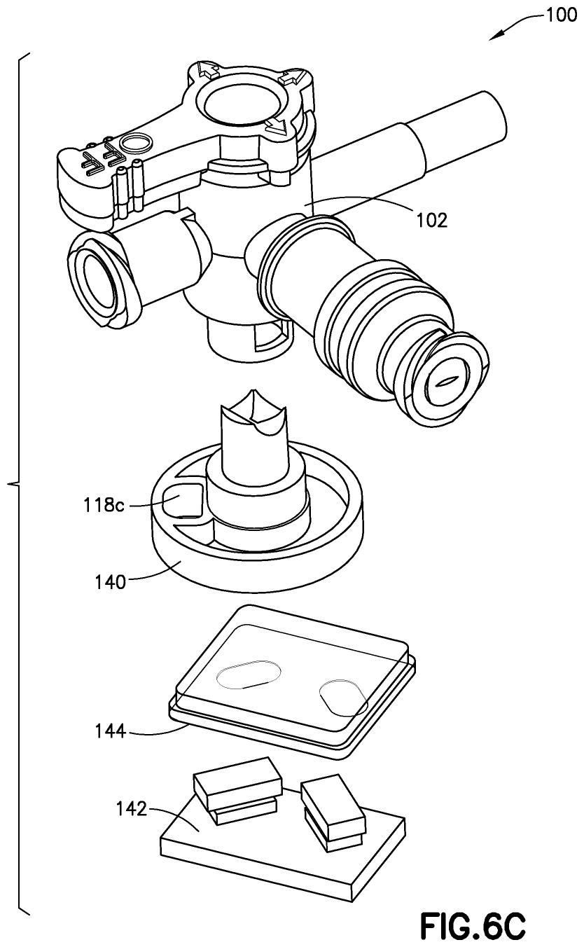

Referring now to FIGS. 6A-6D, in some non-limiting embodiments or aspects, a first portion of valve body 104 extends from top 103a of main body 102, the first portion of valve body 104 extending from top 103a of main body 102 includes handle 114, a second portion of valve body 104 extends from bottom 103b of main body 102 opposite top 103a of main body 102, an end of the second portion of valve body 104 opposite bottom 103b of main body 102 includes disc 140, a face of disc 140 facing opposite a direction toward top 103a of main body 102 includes position structure(s) 118, and/or the position structure(s) 118 includes at least one recessed portion of disc 140. For example, the at least one recessed portion of disc 140 may include single recessed portion 118c.

Medical stopcock assembly 100 may further include circuit board 142 (e.g., a printed circuit board, etc.) including a face facing the face of disc 140 facing opposite the direction toward top 103a of main body 102, and the face of circuit board 142 may include sensor(s) 120. As an example, sensor(s) 120 may include first sensor 120a and second sensor 120b, and first sensor 120a and second sensor 120b may be spaced apart 90 degrees on a plane of circuit board 142. In such an example, cover 144 (e.g., a transparent cover, a glass cover, a cover including openings for first sensor 120a and second sensor 120b, etc.) may be provided on circuit board 142. In some non-limiting embodiments or aspects, housing 122 may house circuit board 142 and cover 144. In some non-limiting embodiment or aspects, circuit board 142 and cover 144 may form all or a part of housing 122.

Depending on a position of valve body 104 relative to main body 102, first sensor 120a may detect single recessed portion 118c or a portion of external surface 116 of valve body 104 (e.g., a bottom of disc 140, etc.) without a recessed portion, and second sensor 120b may detect single recessed portion 118c or a portion of external surface 116 of valve body 104 (e.g., a bottom of disc 140, etc.) without a recessed portion. For example, unique combinations of detections by first sensor 120a and second sensor 120b of single recessed portion 118c and/or a portion of external surface 116 of valve body 104 without a recessed portion may be associated with or correspond to the first position, the second position, or the third position (or the fourth position) of valve body 104 (e.g., FIG. 6C may show single recessed portion 118c in a position corresponding to the second position described herein), and first sensor 120a and second sensor 120b (and/or processor 124, and/or the external computing device) may determine based on a detected unique combination a position of valve body 104 in the first position, the second position, or the third position (or the fourth position).

Although embodiments or aspects have been described in detail for the purpose of illustration and description, it is to be understood that such detail is solely for that purpose and that embodiments or aspects are not limited to the disclosed embodiments or aspects, but, on the contrary, are intended to cover modifications and equivalent arrangements that are within the spirit and scope of the appended claims. For example, it is to be understood that the present disclosure contemplates that, to the extent possible, one or more features of any embodiment or aspect can be combined with one or more features of any other embodiment or aspect. In fact, any of these features can be combined in ways not specifically recited in the claims and/or disclosed in the specification. Although each dependent claim listed below may directly depend on only one claim, the disclosure of possible implementations includes each dependent claim in combination with every other claim in the claim set.

1. A medical stopcock assembly with position detection comprising:
a medical stopcock including:
a main body;
a valve body, wherein the valve body defines a valve passageway and is moveable relative to the main body between a first position where a first input port and an output port are in fluid communication via the valve passageway, a second position where a second input port and the output port are in fluid communication via the valve passageway, and a third position where the second input port and the first input port are in fluid communication via the valve passageway, and wherein an external surface of the valve body includes at least one position structure; and
at least one sensor configured to sense the at least one position structure to determine whether the valve body is in the first position, the second position, or the third position.

2. The medical stopcock assembly of claim 1, wherein a portion of the valve body extends from a top of the main body, wherein the portion of the valve body extending from the top of the main body includes a handle, and wherein the at least one position structure includes at least one recessed portion of the handle.

3. The medical stopcock assembly of claim 2, wherein the at least one recessed portion of the handle includes a first recessed portion and a second recessed portion, and wherein the first recessed portion and the second recessed portion are spaced apart about a circumference of the valve body in a same radial plane of the valve body.

4. The medical stopcock assembly of claim 3, wherein the first recessed portion and the second recessed portion are spaced apart 90 degrees about the circumference of the valve body.

5. The medical stopcock assembly of claim 3, wherein the at least one sensor includes a first sensor and a second sensor, and wherein the first sensor and the second sensor are in the same radial plane of the valve body and a same tangential plane of the valve body.

6. The medical stopcock assembly of claim 1, wherein a first portion of the valve body extends from a top of the main body, wherein the first portion of the valve body extending from the top of the main body includes a handle, wherein a second portion of the valve body extends from a bottom of the main body opposite the top of the main body, wherein the second portion of the valve body includes the at least one position structure, and wherein the at least one position structure includes at least one recessed portion of the valve body.

7. The medical stopcock assembly of claim 6, wherein the at least one recessed portion includes a first recessed portion and a second recessed portion, and wherein the first recessed portion and the second recessed portion are spaced apart about a circumference of the valve body and in an axial direction of the valve body.

8. The medical stopcock assembly of claim 7, wherein the first recessed portion and the second recessed portion are spaced apart 90 degrees about the circumference of the valve body.

9. The medical stopcock assembly of claim 7, wherein the at least one sensor includes a first sensor and a second sensor, and wherein the second sensor is located above the first sensor in the axial direction of the valve body in a same tangential plane of the valve body.

10. The medical stopcock assembly of claim 6, wherein the at least one recessed portion includes a single recessed portion.

11. The medical stopcock assembly of claim 10, wherein the at least one sensor includes a first sensor and a second sensor, and wherein the first sensor and the second sensor are spaced apart about the circumference of the valve body in the same radial plane of the valve body.

12. The medical stopcock assembly of claim 11, wherein the first sensor and the second sensor are spaced apart 90 degrees about the circumference of the valve body in the same radial plane of the valve body.

13. The medical stopcock assembly of claim 1, wherein a first portion of the valve body extends from a top of the main body, wherein the first portion of the valve body extending from the top of the main body includes a handle, wherein a second portion of the valve body extends from a bottom of the main body opposite the top of the main body, wherein an end of the second portion of the valve body opposite the bottom of the main body includes a disc, wherein a face of the disc facing opposite a direction toward the top of the main body includes the at least one position structure, and wherein the at least one position structure includes at least one recessed portion of the disc.

14. The medical stopcock assembly of claim 13, further comprising:
a circuit board including a face facing the face of the disc facing opposite the direction toward the top of the main body, wherein the face of the circuit board includes the at least one sensor.

15. The medical stopcock assembly of claim 14, wherein the at least one sensor includes a first sensor and a second sensor, and wherein the first sensor and the second sensor are spaced apart 90 degrees on a plane of the circuit board.

16. The medical stopcock assembly of claim 14, wherein the at least one sensor is configured to determine a distance to the external surface of the valve body to determine whether the valve body is in the first position, the second position, or the third position.

17. The medical stopcock assembly of claim 16, wherein the at least one sensor includes at least one of a proximity detector, an image sensor, a time-of-flight sensor, or any combination thereof.

18. The medical stopcock assembly of claim 1, further comprising:
a housing including the at least one sensor, wherein the housing holds the at least one sensor in a fixed position when the valve body moves relative to the main body.

19. The medical stopcock assembly of claim 18, wherein the housing is attached to the main body.

20. The medical stopcock assembly of claim 18, wherein the housing includes a wireless communication device configured to communicate, to a computing device, sensor data sensed by the at least one sensor.
